SEQUENCE LISTING (10 sequences)

SEQ ID NO: 1 - DNA, 72 bp, artificial sequence
  oligonucleotide Sdtal1
acgatagtaaaatacttctcgaactcgtcacatatacgtgtacataatgggtaaggaaaagactcacgtttc

SEQ ID NO: 2 - DNA, 87 bp, artificial sequence
  Oligonucleotide Rdtal1
atcaaaagaaacgtgcataaggacatggcctaaattaatatttccgagatacttccttagaaaaactcatcgagcatcaaatgaaac

SEQ ID NO: 3 - DNA, 82 bp, artificial sequence
  Oligonucleotide Sdnqm1
ttgctagcgtaagtcataaaaaataggaaataatcacatatatacaagaaattaaatatgggtaaaaagcctgaactcaccg

SEQ ID NO: 4 - DNA, 72 bp, artificial sequence
  Oligonucleotide Rdnqm1
agtggtatatatatatttatatatataagtaggtacctctactcttaatgattattcctttgccctcggacg

SEQ ID NO: 5 - DNA, 12 bp, artificial sequence
  sequence RBS
aggaggtttgga

SEQ ID NO: 6 - DNA, 22 bp, artificial sequence
  sequence RBS
aacaaaatgaggaggtactgag

SEQ ID NO: 7 - DNA, 16 bp, artificial sequence
  sequence RBS
aagttaagaggcaaga

SEQ ID NO: 8 - DNA, 14 bp, artificial sequence
  sequence RBS
ttcgcagggggaag

SEQ ID NO: 9 - DNA, 18 bp, artificial sequence
  sequence RBS
taagcaggaccggcggcg

SEQ ID NO: 10 - DNA, 12 bp, artificial sequence
  sequence RBS
caccatacactg

(19) United States
(12) Patent Application Publication
(10) Pub. No.: US 2019/0359988 A1
(43) Pub. Date: Nov. 28, 2019

(54) GENETICALLY OPTIMISED MICROORGANISM FOR PRODUCING MOLECULES OF INTEREST

(71) Applicant: ENOBRAQ, RAMONVILLE SAINT-AGNE (FR)

(72) Inventors: Cedric BOISART, Belberaud (FR); Nicolas MORIN, Toulouse (FR)

(21) Appl. No.: 16/480,579

(22) PCT Filed: Jan. 26, 2018
(86) PCT No.: PCT/EP2018/052004

§ 371 Date: Jul. 24, 2019

(30) Foreign Application Priority Data
Jan. 27, 2017 (FR) 1750696

(51) Int. Cl. C12N 15/52; C12P 13/14; C12P 5/02; C12N 9/88; C12N 9/10; C12N 9/12; C12P 7/62

(52) U.S. Cl. CPC C12N 15/52; C12P 13/14; C12P 5/026; C12N 9/88; C12Y 202/01001; C12P 7/625; C12Y 207/01019; C12Y 401/01039; C12N 9/1022; C12N 9/1205; C12Y 202/01002

(57) ABSTRACT

The invention concerns a genetically modified microorganism expressing a functional type I or II RuBisCO enzyme and a functional phosphoribulokinase (PRK), and in which the non-oxidative branch of the pentose phosphate pathway is at least partially inhibited, said microorganism being genetically modified so as to produce an exogenous molecule and/or to overproduce an endogenous molecule. The invention also concerns the use of such a genetically modified microorganism for the production or overproduction of a molecule of interest and processes for the synthesis or bioconversion of molecules of interest.

### FIELD OF THE INVENTION

The invention concerns a genetically modified microorganism, capable of using carbon dioxide as an at least partial carbon source, for the production of molecules of interest. More specifically, the invention relates to a microorganism in which at least the non-oxidative branch of the pentose phosphate pathway is at least partially inhibited. The invention also relates to processes for the production of at least one molecule of interest using such a microorganism.

### STATE OF THE ART

Over the past few years, a number of microbiological processes have been developed to enable the production of molecules of interest in large quantities.

For example, fermentation processes are used to produce molecules by a microorganism from a fermentable carbon source, such as glucose.

Bioconversion processes have also been developed to allow a microorganism to convert a co-substrate, not assimilable by said microorganism, into a molecule of interest. Here again, a carbon source is required, not for the actual production of the molecule of interest, but for the production of cofactors, and more particularly NADPH, that may be necessary for bioconversion. In general, the production yield of such microbiological processes is low, mainly due to the need for cofactors and the difficulty of balancing redox metabolic reactions. There is also the problem of the cost price of such molecules, since a source of carbon assimilable by the microorganism is still necessary. In other words, currently, in order to produce a molecule of interest with a microbiological process, it is necessary to provide a molecule (glucose, or other), certainly of lower industrial value, but which is sufficient to make the production of certain molecules not economically attractive.

At the same time, carbon dioxide (CO₂), whose emissions into the atmosphere are constantly increasing, is used little, if at all, in current microbiological processes, while its consumption by microorganisms for the production of molecules of interest would not only reduce production costs, but also address certain ecological issues.

There is therefore still a need for microbiological processes to enable the production of molecules of interest in large quantities and with lower cost prices than with current processes.

### SUMMARY OF THE INVENTION

The advantage of using non-photosynthetic microorganisms genetically modified to capture CO₂ and use it as the main carbon source, in the same way as plants and photosynthetic microorganisms, has already been demonstrated. For example, microorganisms modified to express a functional RuBisCO (ribulose-1,5-bisphosphate carboxylase/oxygenase—EC 4.1.1.39) and a functional PRK (phosphoribulokinase—EC 2.7.1.19) to reproduce a Calvin cycle and convert ribulose-5-phosphate into two 3-phosphoglycerate molecules by capturing a carbon dioxide molecule have been developed.

By working on the solutions provided by the Calvin cycle to produce molecules of interest using CO₂ as carbon source, the inventors discovered that by coupling part of the Calvin cycle (PRK/RuBisCO) to at least partial inhibition of the non-oxidative branch of the pentose phosphate pathway, it was possible to increase the production yield of molecules of interest. Interestingly, this inhibition, advantageously carried out downstream of the production of ribulose-5-phosphate, promotes the consumption of exogenous CO₂ by the microorganism. The microorganisms thus developed make it possible to produce on a large scale and with an industrially attractive yield a large number of molecules of interest, such as amino acids, organic acids, terpenes, terpenoids, peptides, fatty acids, polyols, etc.

The invention thus relates to a genetically modified microorganism expressing a functional RuBisCO enzyme and a functional phosphoribulokinase (PRK), and in which the non-oxidative branch of the pentose phosphate pathway is at least partially inhibited, said microorganism being genetically modified so as to produce an exogenous molecule of interest and/or to overproduce an endogenous molecule of interest, other than a RuBisCO and/or phosphoribulokinase (PRK) enzyme.

The invention also concerns the use of a genetically modified microorganism according to the invention, for the production or overproduction of a molecule of interest, other than a RuBisCO enzyme and/or a phosphoribulokinase (PRK), preferentially selected from amino acids, peptides, proteins, vitamins, sterols, flavonoids, terpenes, terpenoids, fatty acids, polyols and organic acids.

The present invention also concerns a biotechnological process for producing or overproducing at least one molecule of interest other than a RuBisCO enzyme and/or a phosphoribulokinase (PRK), characterized in that it comprises a step of culturing a genetically modified microorganism according to the invention, under conditions allowing the synthesis or bioconversion, by said microorganism, of said molecule of interest, and optionally a step of recovery and/or purification of said molecule of interest.

It also concerns a process for producing a molecule of interest other than a RuBisCO enzyme and/or a phosphoribulokinase (PRK), comprising (i) inserting at least one sequence encoding an enzyme involved in the synthesis or bioconversion of said molecule of interest in a recombinant microorganism according to the invention, (ii) culturing said microorganism under conditions allowing the expression of said enzyme and optionally (iii) recovering and/or purifying said molecule of interest.

### DESCRIPTION OF THE FIGURES

FIG. 1: Schematic representation of glycolysis, the Entner-Doudoroff pathway and the pentose phosphate pathway, showing the inhibition of the non-oxidative branch of the pentose phosphate pathway, according to the invention;

FIG. 2: Schematic representation of glycolysis and the pentose phosphate pathway, showing the inhibition of the non-oxidative branch of the pentose phosphate pathway and the management of ribulose-5-phosphate by PRK and RuBisCO, according to the invention.

### DETAILED DESCRIPTION OF THE INVENTION

### Definitions

The terms “recombinant microorganism”, “modified microorganism” and “recombinant host cell” are used herein interchangeably and refer to microorganisms that have been genetically modified to express or overexpress endogenous nucleotide sequences, to express heterologous nucleotide sequences, or that have an altered expression of an endogenous gene. “Alteration” means that the expression of the gene, or level of an RNA molecule or equivalent RNA molecules encoding one or more polypeptides or polypeptide subunits, or the activity of one or more polypeptides or polypeptide subunits is regulated, so that the expression, the level or the activity is higher or lower than that observed in the absence of modification.

It is understood that the terms “recombinant microorganism”, “modified microorganism” and “recombinant host cell” refer not only to the particular recombinant microorganism but to the progeny or the potential progeny of such a microorganism. As some modifications may occur in subsequent generations, due to mutation or environmental influences, these offspring may not be identical to the mother cell, but they are still understood within the scope of the term as used here.

In the context of the invention, an at least partially “inhibited” or “inactivated” metabolic pathway refers to an altered metabolic pathway that can no longer function properly in the microorganism considered, compared with the same wild-type microorganism (not genetically modified to inhibit said metabolic pathway). In particular, the metabolic pathway may be interrupted, leading to the accumulation of an intermediate metabolite. Such an interruption may be achieved, for example, by inhibiting the enzyme necessary for the degradation of an intermediate metabolite of the metabolic pathway considered and/or by inhibiting the expression of the gene encoding that enzyme. The metabolic pathway may also be attenuated, i.e. slowed down. Such attenuation may be achieved, for example, by partially inhibiting one or more enzymes involved in the metabolic pathway considered and/or partially inhibiting the expression of a gene encoding at least one of these enzymes and/or by exploiting the cofactors required for certain reactions. The expression “at least partially inhibited metabolic pathway” means that the level of the metabolic pathway considered is reduced by at least 20%, more preferentially at least 30%, 40%, 50%, or more, compared with the level in a wild-type microorganism. The reduction may be greater, and in particular be at least greater than 60%, 70%, 80%, 90%. According to the invention, inhibition may be total, in the sense that the metabolic pathway considered is no longer used at all by said microorganism. According to the invention, such inhibition may be temporary or permanent.

According to the invention, “inhibition of gene expression” means that the gene is no longer expressed in the microorganism considered or that its expression is reduced, compared with wild-type microorganisms (not genetically modified to inhibit gene expression), leading to the absence of production of the corresponding protein or to a significant decrease in its production, and in particular to a decrease of more than 20%, more preferentially 30%, 40%, 50%, 60%, 70%, 80%, 90%. In one embodiment, inhibition can be total, i.e. the protein encoded by said gene is no longer produced at all. Inhibition of gene expression can be achieved by deletion, mutation, insertion and/or substitution of one or more nucleotides in the gene considered. Preferentially, inhibition of gene expression is achieved by total deletion of the corresponding nucleotide sequence. According to the invention, any method of gene inhibition, known per se by the skilled person and applicable to a microorganism, may be used. For example, inhibition of gene expression can be achieved by homologous recombination (Datsenko et al., Proc Natl Acad Sci USA. 2000; 97:6640-5; Lodish et al., Molecular Cell Biology 4^{th} ed. 2000. W. H. Freeman and Company. ISBN 0-7167-3136-3); random or directed mutagenesis to modify gene expression and/or encoded protein activity (Thomas et al., Cell. 1987; 51:503-12); modification of a promoter sequence of the gene to alter its expression (Kaufmann et al., Methods Mol Biol. 2011; 765:275-94. doi: 10.1007/978-1-61779-197-0_16); targeting induced local lesions in genomes (TILLING); conjugation, etc. Another particular approach is gene inactivation by insertion of a foreign sequence, for example by transposon mutagenesis using mobile genetic elements (transposons), of natural or artificial origin. According to another preferred embodiment, inhibition of gene expression is achieved by knock-out techniques. Inhibition of gene expression can also be achieved by extinguishing the gene using interfering, ribozyme or antisense RNA (Daneholt, 2006. Nobel Prize in Physiology or Medicine). In the context of the present invention, the term “interfering RNA” or “iRNA” refers to any iRNA molecule (for example single-stranded RNA or double-stranded RNA) that can block the expression of a target gene and/or facilitate the degradation of the corresponding mRNA. Gene inhibition can also be achieved by genome editing methods that allow direct genetic modification of a given genome, through the use of zinc finger nucleases (Kim et al., PNAS; 93: 1156-1160), transcription activator-like effector nucleases, or “TALEN” (Ousterout et al., Methods Mol Biol. 2016; 1338:27-42. doi: 10.1007/978-1-4939-2932-0_3), a system combining Cas9 nucleases with clustered regularly interspaced short palindromic repeats, or “CRISPR” (Mali et al., Nat Methods. 2013 October; 10(10):957-63. doi: 10.1038/nmeth.2649), or meganucleases (Daboussi et al., Nucleic Acids Res. 2012. 40:6367-79). Inhibition of gene expression can also be achieved by inactivating the protein encoded by said gene.

In the context of the invention, “NADPH-dependent” or “NADPH-consuming” biosynthesis or bioconversion means all biosynthesis or bioconversion pathways in which one or more enzymes require the concomitant supply of electrons obtained by the oxidation of an NADPH cofactor. “NADPH-dependent” biosynthesis or bioconversion pathways notably concern the synthesis of amino acids (e.g. arginine, lysine, methionine, threonine, proline, glutamate, homoserine, isoleucine, valine), terpenoids and terpenes (e.g. farnesene), vitamins and precursors (e.g. pantoate, pantothenate, transneurosporene, phylloquinone, tocopherols), sterols (e.g. squalene, cholesterol, testosterone, progesterone, cortisone), flavonoids (e.g. frambinone, vestinone), organic acids (e.g. coumaric acid, 3-hydroxypropionic acid), polyols (e.g. sorbitol, xylitol, glycerol), polyamines (e.g. spermidine), aromatic molecules from stereospecific hydroxylation, via an NADP-dependent cytochrome p450 (e.g. phenylpropanoids, terpenes, lipids, tannins, fragrances, hormones).

The term “exogenous” as used here in reference to various molecules (nucleotide sequences, peptides, enzymes, etc.) refers to molecules that are not normally or naturally found in and/or produced by the microorganism considered. Conversely, the term “endogenous” or “native” refers to various molecules (nucleotide sequences, peptides, enzymes, etc.), designating molecules that are normally or naturally found in and/or produced by the microorganism considered.

### Microorganisms

The invention proposes genetically modified microorganisms for the production of a molecule of interest, endogenous or exogenous.

“Genetically modified” microorganism means that the genome of the microorganism has been modified to incorporate a nucleic sequence encoding an enzyme involved in the biosynthesis or bioconversion pathway of a molecule of interest, or encoding a biologically active fragment thereof. Said nucleic sequence may have been introduced into the genome of said microorganism or one of its ancestors, by any suitable molecular cloning method. In the context of the invention, the genome of the microorganism refers to all genetic material contained in the microorganism, including extrachromosomal genetic material contained, for example, in plasmids, episomes, synthetic chromosomes, etc. The introduced nucleic sequence may be a heterologous sequence, i.e. one that does not naturally exist in said microorganism, or a homologous sequence. Advantageously, a transcriptional unit with the nucleic sequence of interest is introduced into the genome of the microorganism, under the control of one or more promoters. Such a transcriptional unit also includes, advantageously, the usual sequences such as transcriptional terminators, and, if necessary, other transcription regulatory elements.

Promoters usable in the present invention include constitutive promoters, i.e. promoters that are active in most cellular states and environmental conditions, as well as inducible promoters that are activated or suppressed by exogenous physical or chemical stimuli, and therefore induce a variable state of expression depending on the presence or absence of these stimuli. For example, when the microorganism is a yeast, it is possible to use a constitutive promoter, such as that of a gene among TEF1, TDH3, PGI1, PGK, ADH1. Examples of inducible promoters that can be used in yeast are tetO-2, GAL10, GAL10-CYC1, PHO5.

In general, the genetically modified microorganism according to the invention has the following features:
Expression of a functional RuBisCO (EC 4.1.1.39);
Expression of a functional PRK (EC 2.7.1.19);
At least partial inhibition of the non-oxidative branch of the pentose phosphate pathway; and
Expression of at least one gene involved in the synthesis and/or bioconversion of a molecule of interest, and/or inhibition of at least one gene encoding activity competing with the synthesis and/or bioconversion of a molecule of interest.

According to the invention, any microorganism can be used. Preferably the microorganism is a eukaryotic cell, preferentially selected from yeasts, fungi, microalgae, or a prokaryotic cell, preferentially a bacterium or cyanobacterium.

In one embodiment, the genetically modified microorganism according to the invention is a yeast, preferentially selected from among the ascomycetes (Spermophthoraceae and Saccharomycetaceae), basidiomycetes (Leucosporidium, Rhodosporidium, Sporidiobolus, Filobasidium, and Filobasidiella) and deuteromycetes yeasts belonging to Fungi imperfecti (Sporobolomycetaceae, and Cryptococcaceae). Preferentially, the genetically modified yeast according to the invention belongs to the genus Pichia, Kluyveromyces, Saccharomyces, Schizosaccharomyces, Candida, Lipomyces, Rhodotorula, Rhodosporidium, Yarrowia, or Debaryomyces. More preferentially, the genetically modified yeast according to the invention is selected from Pichia pastoris, Kluyveromyces lactis, Kluyveromyces marxianus, Saccharomyces cerevisiae, Saccharomyces carlsbergensis, Saccharomyces diastaticus, Saccharomyces douglasii, Saccharomyces kluyveri, Saccharomyces norbensis, Saccharomyces oviformis, Schizosaccharomyces pombe, Candida albicans, Candida tropicalis, Rhodotorula glutinis, Rhodosporidium toruloides, Yarrowia lipolytica, Debaryomyces hansenii and Lipomyces starkeyi.

In another embodiment, the genetically modified microorganism according to the invention is a fungus, and more particularly a “filamentous” fungus. In the context of the invention, “filamentous fungi” refers to all filamentous forms of subdivision Eumycotina. For example, the genetically modified fungus according to the invention belongs to the genus Aspergillus, Trichoderma, Neurospora, Podospora, Endothia, Mucor, Cochliobolus or Pyricularia. Preferentially, the genetically modified fungus according to the invention is selected from Aspergillus nidulans, Aspergillus niger, Aspergillus awomari, Aspergillus oryzae, Aspergillus terreus, Neurospora crassa, Trichoderma reesei, and Trichoderma viride.

In another embodiment, the genetically modified microorganism according to the invention is a microalga. In the context of the invention, “microalga” refers to all eukaryotic microscopic algae, preferentially belonging to the classes or superclasses Chlorophyceae, Chrysophyceae, Prymnesiophyceae, Diatomae or Bacillariophyta, Euglenophyceae, Rhodophyceae, or Trebouxiophyceae. Preferentially, the genetically modified microalgae according to the invention are selected from Nannochloropsis sp. (e.g. Nannochloropsis oculata, Nannochloropsis gaditana, Nannochloropsis salina), Tetraselmis sp. (e.g. Tetraselmis suecica, Tetraselmis chuii), Chlorella sp. (e.g. Chlorella salina, Chlorella protothecoides, Chlorella ellipsoidea, Chlorella emersonii, Chlorella minutissima, Chlorella pyrenoidosa, Chlorella sorokiniana, Chlorella vulgaris), Chlamydomonas sp. (e.g. Chlamydomonas reinhardtii) Dunaliella sp. (e.g. Dunaliella tertiolecta, Dunaliella salina), Phaeodactulum tricornutum, Botrycoccus braunii, Chroomonas salina, Cyclotella cryptica, Cyclotella sp., Ettlia texensis, Euglena gracilis, Gymnodinium nelsoni, Haematococcus pluvialis, Isochrysis galbana, Monoraphidium minutum, Monoraphidium sp, Neochloris oleoabundans, Nitzschia laevis, Onoraphidium sp., Pavlova lutheri, Phaeodactylum tricornutum, Porphyridium cruentum, Scenedesmus sp. (e.g. Scenedesmus obliquuus, Scenedesmus quadricaulaula, Scenedesmus sp.), Stichococcus bacillaris, Spirulina platensis, Thalassiosira sp.

In one embodiment, the genetically modified microorganism according to the invention is a bacterium, preferentially selected from phyla Acidobacteria, Actinobacteria, Aquificae, Bacterioidetes, Chlamydia, Chlorobi, Chloroflexi, Chrysiogenetes, Cyanobacteria, Deferribacteres, Deinococcus-Thermus, Dictyoglomi, Fibrobacteres, Firmicutes, Fusobacteria, Gemmatimonadetes, Nitrospirae, Planctomycetes, Proteobacteria, Spirochaetes, Thermodesulfobacteria, Thermomicrobia, Thermotogae, or Verrucomicrobia. Preferably, the genetically modified bacterium according to the invention belongs to the genus Acaryochloris, Acetobacter, Actinobacillus, Agrobacterium, Alicyclobacillus, Anabaena, Anacystis, Anaerobiospirillum, Aquifex, Arthrobacter, Arthrospira, Azobacter, Bacillus, Brevibacterium, Burkholderia, Chlorobium, Chromatium, Chlorobaculum, Clostridium, Corynebacterium, Cupriavidus, Cyanothece, Enterobacter, Deinococcus, Erwinia, Escherichia, Geobacter, Gloeobacter, Gluconobacter, Hydrogenobacter, Klebsiella, Lactobacillus, Lactococcus, Mannheimia, Mesorhizobium, Methylobacterium, Microbacterium, Microcystis, Nitrobacter, Nitrosomonas, Nitrospina, Nitrospira, Nostoc, Phormidium, Prochlorococcus, Pseudomonas, Ralstonia, Rhizobium, Rhodobacter, Rhodococcus, Rhodopseudomonas, Rhodospirillum, Salmonella, Scenedesmun, Serratia, Shigella, Staphylococcus, Streptomyces, Synechoccus, Synechocystis, Thermosynechococcus, Trichodesmium, or Zymomonas. Also preferably, the genetically modified bacterium according to the invention is selected from the species Agrobacterium tumefaciens, Anaerobiospirillum succiniciproducens, Actinobacillus succinogenes, Aquifex aeolicus, Aquifex pyrophilus, Bacillus subtilis, Bacillus amyloliquefacines, Brevibacterium ammoniagenes, Brevibacterium immariophilum, Clostridium pasteurianum, Clostridium ljungdahlii, Clostridium acetobutylicum, Clostridium beigerinckii, Corynebacterium glutamicum, Cupriavidus necator, Cupriavidus metallidurans, Enterobacter sakazakii, Escherichia coli, Gluconobacter oxydans, Hydrogenobacter thermophilus, Klebsiella oxytoca, Lactococcus lactis, Lactobacillus plantarum, Mannheimia succiniciproducens, Mesorhizobium loti, Pseudomonas aeruginosa, Pseudomonas mevalonii, Pseudomonas pudica, Pseudomonas putida, Pseudomonas fluorescens, Rhizobium etli, Rhodobacter capsulatus, Rhodobacter sphaeroides, Rhodospirillum rubrum, Salmonella enterica, Salmonella enterica, Salmonella typhi, Salmonella typhimurium, Shigella dysenteriae, Shigella flexneri, Shigella sonnei, Staphylococcus aureus, Streptomyces coelicolor, Zymomonas mobilis, Acaryochloris marina, Anabaena variabilis, Arthrospira platensis, Arthrospira maxa, Chlorobium tepidum, Chlorobaculum sp., Cyanothece sp., Gloeobacter violaceus, Microcystis aeruginosa, Nostoc punctiforme, Prochlorococcus marinus, Synechococcus elongatus, Synechocystis sp., The rmosynechococcus elongatus, Trichodesmium erythraeum, and Rhodopseudomonas palustris.

### Expression of a Functional RuBisCO and a Functional PRK

According to the invention, the microorganism can naturally express a functional RuBisCO and a functional PRK. This is the case, for example, for photosynthetic microorganisms such as microalgae and cyanobacteria.

There are several forms of RuBisCO in nature (Tabita et al., J Exp Bot. 2008; 59(7):1515-24. doi: 10.1093/jxb/erm361). Forms I, II and III catalyze the carboxylation and oxygenation reactions of ribulose-1,5-biphosphate. Form I is present in eukaryotes and bacteria. It consists of two types of subunits: large subunits (RbcL) and small subunits (RbcS). The functional enzyme complex is a hexadecamer consisting of eight L subunits and eight S subunits. The correct assembly of these subunits also requires the intervention of at least one specific chaperone: RbcX (Liu et al., Nature. 2010 Jan. 14; 463(7278):197-202. doi: 10.1038/nature08651). Form II is mainly found in proteobacteria, archaea and dinoflagellate algae. Its structure is much simpler: it is a homodimer (formed by two identical RbcL subunits). Depending on the organism, the genes encoding a type I RuBisCO may be called rbcL/rbcS (for example Synechococcus elongatus), or cbxLC/cbxSC, cfxLC/cfxSC, cbbL/cbbS (for example Cupriavidus necator). Depending on the organism, the genes encoding a type II RuBisCO are generally called cbbM (for example Rhodospirillum rubrum). Form III is present in the archaea. It is generally found in the form of dimers of the RbcL subunit, or in pentamers of dimers. Depending on the organism, the genes encoding a type III RuBisCO may be called rbcL (for example Thermococcus kodakarensis), cbbL (for example Haloferax sp.).

Two classes of PRKs are known: class I enzymes found in proteobacteria are octamers, while class II enzymes found in cyanobacteria and plants are tetramers or dimers. Depending on the organism, the genes encoding a PRK may be called prk (for example Synechococcus elongatus), prkA (for example Chlamydomonas reinhardtii), prkB (for example Escherichia coli), prk1, prk2 (for example Leptolyngbya sp.), cbbP (for example Nitrobacter vulgaris) or cfxP (for example Cupriavidus necator).

In the case where the microorganism used does not naturally express a functional RuBisCO and a functional PRK, said microorganism is genetically modified to express heterologous RuBisCO and PRK. Advantageously, in such a case, the microorganism is transformed so as to integrate into its genome one or more expression cassettes integrating the sequences encoding said proteins, and advantageously the appropriate transcription factors. Depending on the type of RuBisCO to be expressed, it may also be necessary to have one or more chaperone proteins expressed by the microorganism, in order to promote the proper assembly of the subunits forming the RuBisCO. This is particularly the case for type I RuBisCO, where the introduction and expression of genes encoding a specific chaperone (Rbcx) and generalist chaperones (GroES and GroEL, for example) are necessary to obtain a functional RuBisCO. Application WO2015/107496 describes in detail how to genetically modify a yeast to express a functional type I RuBisCO and PRK. It is also possible to refer to the method described in GUADALUPE-MEDINA et al. (Biotechnology for Biofuels, 6, 125, 2013).

In one embodiment, the microorganism is genetically modified to express a type I RuBisCO. In another embodiment, the microorganism is genetically modified to express a type II RuBisCO. In another embodiment, the microorganism is genetically modified to express a type III RuBisCO.

Tables 1 and 2 below list, as examples, sequences encoding RuBisCO and PRK that can be used to transform a microorganism to express a functional RuBisCO and a functional PRK.

TABLE 1
Examples of sequences encoding a RuBisCO

| Gene | GenBank | GI | Organism |
|---|---|---|---|
| rbcL | BAD78320.1 | 56685098 | Synechococcus elongatus |
| rbcS | BAD78319.1 | 56685097 | Synechococcus elongatus |
| cbbL2 | CAJ96184.1 | 113529837 | Cupriavidus necator |
| cbbS | P09658.2 | 6093937 | Cupriavidus necator |
| cbbM | P04718.1 | 132036 | Rhodospirillum rubrum |
| cbbM | Q21YM9.1 | 115502580 | Rhodoferax ferrireducens |
| cbbM | Q479W5.1 | 115502578 | Dechloromonas aromatica |
| rbcL | O93627.5 | 37087684 | Thermococcus kodakarensis |
| cbbL | CQR50548.1 | 811260688 | Haloferox sp. Arc-Hr |

TABLE 2
Examples of sequences encoding a PRK

| Gene | GenBank | GI | Organism |
|---|---|---|---|
| Prk | BAD78757.1 | 56685535 | Synechococcus elongatus |
| cfXP | P19923.3 | 125575 | Cupriavidus necator |
| PRK | P09559.1 | 125579 | Spinacia oleracea |
| cbbP | P37100.1 | 585367 | Nitrobacter vulgaris |

### Inhibition of the Non-Oxidative Branch of the Pentose Phosphate Pathway

According to the invention, the non-oxidative branch of the pentose phosphate pathway is at least partially inhibited, so that the microorganism is no longer able to join the glycolysis pathway through the pentose phosphate pathway.

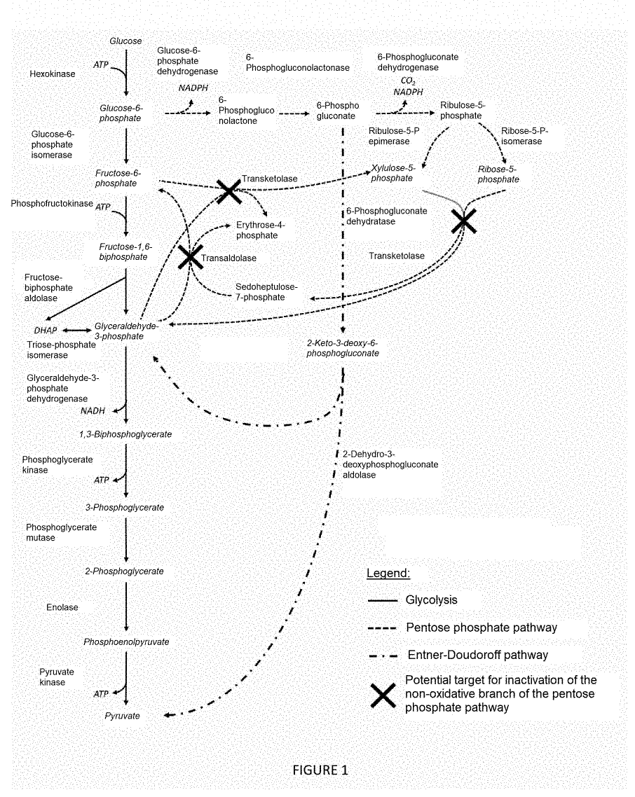

Preferentially, the microorganism is genetically modified to inhibit the non-oxidative branch of the pentose phosphate pathway downstream of ribulose-5-phosphate production (FIG. 1).

The interruption of the non-oxidative branch of the pentose phosphate pathway downstream of ribulose-5-phosphate (Ru5P) production is advantageously achieved by at least partial inhibition of a transaldolase (E.C. 2.2.2.1.2) normally produced by the microorganism.

Transaldolase is an enzyme that catalyzes a transferase-type reaction between the metabolite pairs sedoheptulose 7-phosphate/glyceraldehyde 3-phosphate and erythrose-4-phosphate/fructose 6-phosphate.

Depending on the organism, the genes encoding transaldolase may be called tal, talA, talB (for example in Escherichia coli, Synechocystis sp.), TALDO, TALDO1, TALDOR (for example in Homo sapiens, Mus musculus), TAL1 (for example in Saccharomyces cerevisiae), TAL2 (for example in Nostoc punctiforme), talA1, talA2 (for example Streptococcus gallolyticus), talB1, talB2 (for example Azotobacter vinelandii), or NQM1 (for example in Saccharomyces cerevisiae).

Alternatively or additionally, the interruption of the non-oxidative branch of the pentose phosphate pathway downstream of ribulose-5-phosphate (Ru5P) production can be obtained by at least partial inhibition of a transketolase (E.C. 2.2.2.1.1) normally produced by the microorganism.

Transketolase is an enzyme that catalyzes a transferase reaction between the metabolite pairs sedoheptulose-7-phosphate/glyceraldehyde 3-phosphate, and ribose-5-phosphate/xylulose-5-phosphate, as well as between the pairs fructose-6-phosphate/glyceraldehyde 3-phosphate, and erythrose-4-phosphate/xylulose-5-phosphate. Depending on the organism, the genes encoding transketolase may be called TKL, TKL1, TKL2 (for example Saccharomyces cerevisiae), tklA, tklB (for example Rhodobacter sphaeroides), tktA, tktB, (for example Escherichia coli), TKT, TKT1, TKT2 (for example Homo sapiens, Dictyostelium discoideum), or TKTL1, TKTL2 (for example Bos taurus), or cbbT, cbbTC, cbbTP (for example Cupriavidus necator, Synechococcus sp.).

In one particular example, the microorganism is genetically modified so that the expression of the gene encoding transaldolase is at least partially inhibited. Preferentially, gene expression is completely inhibited. Alternatively or additionally, the microorganism is genetically modified so that the expression of the gene encoding transketolase is at least partially inhibited. Preferentially, gene expression is completely inhibited.

Tables 3 and 4 below list, as examples, the sequences encoding a transaldolase or a transketolase, which can be inhibited depending on the target microorganism. The skilled person knows which gene corresponds to the enzyme of interest to be inhibited depending on the microorganism.

TABLE 3
Examples of sequences encoding a transaldolase

| Gene | GenBank | GI | Organism |
|---|---|---|---|
| TAL1 | P15019.4 | 1729825 | Saccharomyces cerevisiae |
| NQM1 | P53228.1 | 1729826 | Saccharomyces cerevisiae |
| talA | BAA21821.1 | 2337774 | Escherichia coli |
| talB | BAA16812.1 | 1651885 | Synechocystis sp. |

TABLE 4
Examples of sequences encoding a transketolase

| Gene | GenBank | GI | Organism |
|---|---|---|---|
| TKL1 | NP_015399.1 | 6325331 | Saccharomyces cerevisiae |
| TKL2 | NP_009675.3 | 398364879 | Saccharomyces cerevisiae |
| tktA | AAA69102.1 | 882464 | Escherichia coli |
| cbbT | AHF62567.1 | 572996306 | Synechococcus sp. |

In general, the junction between the pentose phosphate pathway and the glycolysis pathway is no longer possible through the non-oxidative branch of the pentose phosphate pathway, or at least significantly decreased, in the genetically modified microorganism according to the invention.

In a particular exemplary embodiment, the microorganism is a yeast of the genus Saccharomyces cerevisiae in which the expression of the NQM1 and/or TAL1 gene is at least partially inhibited.

In another particular exemplary embodiment, the microorganism is a bacterium of the genus Escherichia coli in which the expression of the talA gene is at least partially inhibited.

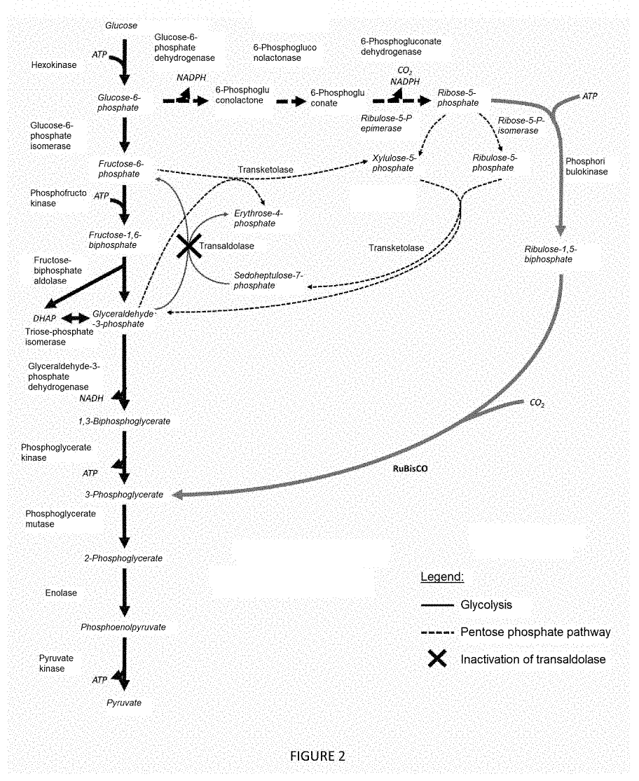

According to the invention, the genetically modified microorganism, which expresses a functional RuBisCO and a functional PRK, and whose non-oxidative branch of the pentose phosphate pathway is at least partially inhibited, is no longer able to join the glycolysis pathway via pentose phosphates. On the other hand, it is capable of producing glyceraldehyde-3-phosphate (G3P) from Ru5P synthesized by the oxidative branch of the pentose phosphate pathway, via the heterologous expression of PRK and RuBisCO, while fixing an additional carbon molecule (FIG. 2).

Thus, the genetically modified microorganism is able to produce NADPH via the oxidative branch of the pentose phosphate pathway, and G3P via the heterologous expression of PRK and RuBisCO, using exogenous CO₂, and in particular atmospheric CO₂, as complementary carbon source.

Thus, the genetically modified microorganism according to the invention makes it possible to increase carbon yield, by fixing and using exogenous CO₂, for the production of NADPH and G3P (and subsequently molecules of interest). Here again, there is an increase in carbon yield.

### Inhibition of the Entner-Doudoroff Pathway

In one particular embodiment, the genetically modified microorganism according to the invention has an Entner-Doudoroff pathway, and this is at least partially inhibited. This pathway, mainly found in bacteria (especially Gram-negative bacteria), is an alternative to glycolysis and the pentose pathway for the production of pyruvate from glucose. More precisely, this pathway connects to the pentose phosphate pathway at P-gluconate to supply glycolysis, particularly at pyruvate.

Preferentially, the microorganism is genetically modified to inhibit Entner-Doudoroff pathway reactions downstream of 6-phosphogluconate production. This inhibition eliminates a possible competing pathway, and ensures the availability of 6-phosphogluconate as a substrate for PRK/RuBisCO engineering.

The interruption of the Entner-Doudoroff pathway downstream of 6-phosphogluconate production specifically targets one or more reactions in the pyruvate synthesis process from 6-phosphogluconate. This synthesis is initiated by the successive actions of two enzymes: (i) 6-phosphogluconate dehydratase (“EDD”—EC. 4.2.1.12), and (ii) 2-dehydro-3-deoxy-phosphogluconate aldolase (“EDA”—E.C. 4.1.2.14).

6-Phosphogluconate dehydratase catalyzes the dehydration of 6-phosphogluconate to 2-keto-3-deoxy-6-phosphogluconate. Depending on the organism, the genes encoding 6-phosphogluconate dehydratase may be called edd (GenBank NP_416365, for example, in Escherichia coli), or ilvD (for example, in Mycobacterium sp.).

2-Dehydro-3-deoxy-phosphogluconate aldolase catalyzes the synthesis of a pyruvate molecule and a glyceraldehyde-3-phosphate molecule from the 2-keto-3-deoxy-6-phosphogluconate produced by 6-phosphogluconate dehydratase. Depending on the organism, the genes encoding 2-dehydro-3-deoxy-phosphogluconate aldolase may be called eda (GenBank NP_416364, for example, in Escherichia coli), or kdgA (for example in Thermoproteus tenax), or dgaF (for example in Salmonella typhimurium).

In one particular example, the microorganism is genetically modified so that the expression of the gene encoding 6-phosphogluconate dehydratase is at least partially inhibited. Preferentially, gene expression is completely inhibited.

Alternatively or additionally, the microorganism is genetically modified so that the expression of the gene encoding 2-dehydro-3-deoxy-phosphogluconate aldolase is at least partially inhibited. Preferentially, gene expression is completely inhibited.

Tables 5 and 6 below list, as examples, the sequences encoding a 6-phosphogluconate dehydratase and a 2-dehydro-3-deoxy-phosphogluconate aldolase that can be inhibited depending on the target microorganism. The skilled person knows which gene corresponds to the enzyme of interest to be inhibited depending on the microorganism.

TABLE 5
Examples of sequences encoding an EDD

| Gene | GenBank | GI | Organism |
|---|---|---|---|
| edd | NP_416365.1 | 16129804 | Escherichia coli |
| ilvD | CND70554.1 | 893638835 | Mycobacterium tuberculosis |
| edd | AJQ65426.1 | 764046652 | Salmonella enterica |

TABLE 6
Examples of sequences encoding an EDA

| Gene | GenBank | GI | Organism |
|---|---|---|---|
| eda | AKF72280.1 | 817591701 | Escherichia coli |
| kdgA | Q704D1.1 | 74500902 | Thermoproteus tenax |
| eda | O68283.2 | 81637643 | Pseudomonas aeruginosa |

In general, in this embodiment, pyruvate production is no longer possible via the Entner-Doudoroff pathway, or at least significantly reduced.

In a particular exemplary embodiment, the microorganism is a bacterium of the genus Escherichia coli in which the expression of the edd gene is at least partially inhibited.

In one particular example, the bacterium of the genus Escherichia coli is genetically modified so that the expression of the talA and edd genes are at least partially inhibited.

According to the invention, the genetically modified microorganism, which expresses a functional RuBisCO and a functional PRK, and whose non-oxidative branch of the pentose phosphate pathway and the Entner-Doudoroff pathway are at least partially inhibited, is no longer capable of producing pyruvate by the Entner-Doudoroff pathway or the pentose phosphate pathway. The carbon flux from glucose during the production of NADPH is therefore preferably directed towards the PRK/RuBisCO engineering.

### Production of Molecules of Interest

According to the invention, the genetically modified microorganism is transformed so as to produce an exogenous molecule of interest and/or to overproduce an endogenous molecule of interest.

In the context of the invention, molecule of interest preferentially refers to a small organic molecule with a molecular mass less than or equal to 0.8 kDa.

In general, genetic modifications made to the microorganism, as described above, improve the carbon yield of the synthesis and/or bioconversion pathways of molecules of interest.

In the context of the invention, “improved” yield refers to the quantity of the finished product. In general, in the context of the invention, the carbon yield corresponds to the ratio of quantity of finished product to quantity of fermentable sugar, particularly by weight. According to the invention, the carbon yield is increased in the genetically modified microorganisms according to the invention, compared with wild-type microorganisms, placed under identical culture conditions. Advantageously, the carbon yield is increased by 2%, 5%, 10%, 15%, 18%, 20%, or more. The genetically modified microorganism according to the invention may produce a larger quantity of molecules of interest (finished product) than heterologous molecules produced by a genetically modified microorganism simply to produce or overproduce that molecule. According to the invention, the genetically microorganism may also overproduce an endogenous molecule compared with the wild-type microorganism. The overproduction of an endogenous molecule is mainly understood in terms of quantities. Advantageously, the genetically modified microorganism produces at least 20%, 30%, 40%, 50%, or more by weight of the endogenous molecule than the wild-type microorganism. Advantageously, the microorganism according to the invention is genetically modified so as to produce or overproduce at least one molecule among amino acids, terpenoids, terpenes, vitamins and/or vitamin precursors, sterols, flavonoids, organic acids, polyols, polyamines, aromatic molecules obtained from stereospecific hydroxylation, via an NADP-dependent cytochrome p450, etc.

In one particular example, the microorganism is genetically modified to overproduce at least one amino acid, preferentially selected from arginine, lysine, methionine, threonine, proline, glutamate, homoserine, isoleucine and valine.

In one particular example, the microorganism is genetically modified to produce or overproduce molecules from the terpenoid pathway, such as farnesene, and from the terpene pathway.

In one particular example, the microorganism is genetically modified to produce or overproduce a vitamin or precursor, preferentially selected from pantoate, pantothenate, transneurosporene, phylloquinone and tocopherols.

In one particular example, the microorganism is genetically modified to produce or overproduce a sterol, preferentially selected from squalene, cholesterol, testosterone, progesterone and cortisone.

In one particular example, the microorganism is genetically modified to produce or overproduce a flavonoid, preferentially selected from frambinone and vestinone.

In one particular example, the microorganism is genetically modified to produce or overproduce an organic acid, preferentially selected from coumaric acid and 3-hydroxypropionic acid.

In one particular example, the microorganism is genetically modified to produce or overproduce a polyol, preferentially selected from sorbitol, xylitol and glycerol.

In one particular example, the microorganism is genetically modified to produce or overproduce a polyamine, preferentially spermidine.

In one particular example, the microorganism is genetically modified to produce or overproduce an aromatic molecule from a stereospecific hydroxylation, via an NADP-dependent cytochrome p450, preferentially selected from phenylpropanoids, terpenes, lipids, tannins, fragrances, hormones.

In the case where the molecule of interest is obtained by bioconversion, the genetically modified microorganism is advantageously cultured in a culture medium including the substrate to be converted. In general, the production or overproduction of a molecule of interest by a genetically modified microorganism according to the invention is obtained by culturing said microorganism in an appropriate culture medium known to the skilled person.

The term “appropriate culture medium” generally refers to a sterile culture medium providing essential or beneficial nutrients for the maintenance and/or growth of said microorganism, such as carbon sources; nitrogen sources such as ammonium sulfate; sources of phosphors, for example, potassium phosphate monobasic; trace elements, for example, salts of copper, iodide, iron, magnesium, zinc or molybdate; vitamins and other growth factors such as amino acids or other growth promoters. An antifoam agent can be added as needed. According to the invention, this appropriate culture medium may be chemically defined or complex. The culture medium may thus be identical or similar in composition to a synthetic medium, as defined by Verduyn et al. (Yeast. 1992. 8:501-17), adapted by Visser et al. (Biotechnology and bioengineering. 2002. 79:674-81), or commercially available such as yeast nitrogen base (YNB) medium (MP Biomedicals or Sigma-Aldrich).

In particular, the culture medium may include a simple carbon source, such as glucose, galactose, sucrose, molasses, or the by-products of these sugars, optionally supplemented with CO₂ as carbon co-substrate. According to the present invention, the simple carbon source must allow the normal growth of the microorganism of interest. It is also possible, in some cases, to use a complex carbon source, such as lignocellulosic biomass, rice straw, or starch. The use of a complex carbon source usually requires pretreatment before use.

In one particular embodiment, the culture medium contains at least one carbon source among monosaccharides such as glucose, xylose or arabinose, disaccharides such as sucrose, organic acids such as acetate, butyrate, propionate or valerate to promote different kinds of polyhydroxyalkanoate (PHA), treated or untreated glycerol.

Depending on the molecules to be produced and/or overproduced, it is possible to exploit the supply of nutritional factors (N, O, P, S, K+, Mg2+, Fe2+, Mn, Co, Cu, Ca, Sn; Koller et al., Microbiology Monographs, G.-Q. Chen, 14: 85-119, (2010)). This is particularly the case to promote the synthesis and intracellular accumulation of PHA including PHB.

According to the invention, any culture method allowing the production on an industrial scale of molecules of interest can be considered. Advantageously, the culture is done in bioreactors, especially in batch, fed-batch and/or continuous culture mode. Preferentially, the culture associated with the production of the molecule of interest is in fed-batch mode corresponding to a controlled supply of one or more substrates, for example by adding a concentrated glucose solution whose concentration can be between 200 g/L and 700 g/L. A controlled supply of vitamins during the process can also be beneficial to productivity (Alfenore et al., Appl Microbiol Biotechnol. 2002. 60:67-72). It is also possible to add an ammonium salt solution to limit the nitrogen supply.

Fermentation is generally carried out in bioreactors, with possible steps of solid and/or liquid precultures in Erlenmeyer flasks, with an appropriate culture medium containing at least a simple carbon source and/or an exogenous CO₂ supply, necessary for the production of the molecule of interest.

In general, the culture conditions of the microorganisms according to the invention are easily adaptable by the skilled person, depending on the microorganism and/or the molecule to be produced/overproduced. For example, the culture temperature is between 20° C. and 40° C. for yeasts, preferably between 28° C. and 35° C., and more particularly around 30° C., for S. cerevisiae. The culture temperature is between 25° C. and 35° C., preferably 30° C., for Cupriavidus necator.

The invention therefore also relates to the use a genetically modified microorganism according to the invention, for the production or overproduction of a molecule of interest, other than a RuBisCO enzyme and/or a phosphoribulokinase (PRK), and preferentially selected from amino acids, peptides, proteins, vitamins, sterols, flavonoids, terpenes, terpenoids, fatty acids, polyols and organic acids.

The invention also relates to a biotechnological process for producing at least one molecule of interest other than a RuBisCO enzyme and/or a phosphoribulokinase (PRK), characterized in that it comprises a step of culturing a genetically modified microorganism according to the invention, under conditions allowing the synthesis or bioconversion, by said microorganism, of said molecule of interest, and optionally a step of recovering and/or purifying said molecule of interest.

In one particular embodiment, the microorganism is genetically modified to express at least one enzyme involved in the synthesis of said molecule of interest.

In another particular embodiment, the microorganism is genetically modified to express at least one enzyme involved in the bioconversion of said molecule of interest.

The invention also relates to a process for producing a molecule of interest comprising (i) inserting at least one sequence encoding an enzyme involved in the synthesis or bioconversion of said molecule of interest into a recombinant microorganism according to the invention, (ii) culturing said microorganism under conditions allowing the expression of said enzyme and optionally (iii) recovering and/or purifying said molecule of interest.

For example, it is possible to produce farnesene by a yeast, such as a yeast of the genus Saccharomyces cerevisiae, genetically modified to express functional PRK and RuBisCO, a farnesene synthase and in which the expression of a TAL1 gene (Gene ID: 851068) is at least partially inhibited.

It is also possible to overproduce glutamate by a bacterium, such as a bacterium of the genus Escherichia coli, genetically modified to express functional PRK and RuBisCO, and in which the expression of the talA (Gene ID: 947006.) and sucA (Gene ID: 945303) genes is at least partially inhibited.

### EXAMPLES

### Example 1: Bioinformatics Analysis

a) Comparison of Carbon Fixation Yields from Glucose Between a Wild-Type Strain Using the Pentose Phosphate Pathway and Glycolysis and a Modified Strain According to the Invention

In order to evaluate the benefit of the modifications according to the invention, theoretical yield calculations were carried out on the basis of the stoichiometry of the reactions involved.

Two cases were analyzed: (i) a wild-type strain, using the pentose phosphate pathway to supply a NADPH-dependent biosynthetic pathway (for example farnesene synthesis), and (ii) a modified strain according to the invention, under the same conditions.

In the context of the improvement of NADPH-dependent biosynthetic pathways, the theoretical balance of the formation of NADPH and glyceraldehyde-3-phosphate (G3-P) from glucose via the pentose phosphate pathway was calculated according to the following equation (1):

3Glucose+5ATP+6NADP⁺+3H₂O→5G3-P+5ADP+6NADPH+11H⁺+3CO₂ (1)

Going down to pyruvate formation from G3P, we arrive at the following balance:

3Glucose+5ADP+6NADP⁺+5NAD⁺+5Pᵢ→5Pyruvate+5ATP+6NADPH+5NADH+11H⁺+3CO₂+2H₂O (2)

If we normalize the balance for one mole of glucose, we obtain the following yield:

Glucose+1.67ADP+2NADP⁺+1.67NAD⁺+1.67Pᵢ→1.67Pyruvate+1.67ATP+2NADPH+1.67NADH+3.67H⁺+CO₂+0.67H₂O (3)

Through the pentose pathway, 1.67 moles of pyruvate and 2 moles of NADPH are produced from one mole of glucose. However, one mole of carbon is lost by decarboxylation during the formation of ribulose-5-phosphate by 6-phosphogluconate dehydrogenase (EC 1.1.1.44).

The maximum theoretical pyruvate production yield when producing 2 NADPH by the pentose phosphate pathway is therefore 0.82 g_{pyruvate}/g_{glucose} (g of synthesized pyruvate, per glucose consumed).

By integrating PRK/RuBisCO engineering into a strain inhibited for the non-oxidative branch of the pentose phosphate pathway (for example ΔTAL1-ΔNQM1 in S. cerevisiae yeast), the carbon fixation flux is redirected from the oxidative branch of the pentose phosphate pathway to the PRK/RuBisCO engineering (FIG. 2). This flux is related to the end of the glycolysis pathway, at the level of 3-phosphoglycerate (3PG) formation, with the following yield:

Glucose+2ATP+2NADP⁺+2H₂O→2 3PG+2ADP+2NADPH+6H⁺ (5)

Going down to pyruvate formation from 3PG, we arrive at the following balance:

Glucose+2NADP⁺→2Pyruvate+2NADPH+4H⁺ (6)

The integration of the modifications according to the invention makes it possible to recover the carbon molecule otherwise lost by decarboxylation in the pentose phosphate pathway. The maximum theoretical yield of pyruvate synthesis during the production of 2 NADPH by the engineering is therefore 0.98 g_{pyruvate}/g_{glucose}, which allows a 20.5% improvement over that obtained by the pentose phosphate pathway.

### b) Simulation of Biosynthesis Yields by Flux Balance Analysis

In a bioinformatics approach, flux balance analyses (FBAs) were also performed to simulate the impact of the modifications described according to the invention on the yield of different biosynthetic pathways.

FBAs are based on mathematical models that simulate metabolic networks at the genome scale (Orth et al., Nat Biotechnol. 2010; 28: 245-248). Reconstructed networks contain the known metabolic reactions of a given organism and integrate the needs of the cell, in particular to ensure cell maintenance or growth. FBAs make it possible to calculate the flow of metabolites through these networks, making it possible to predict theoretical growth rates as well as metabolite production yields.

### i) Procedure

FBA simulations were performed with the OptFlux software (Rocha et al., BMC Syst Biol. 2010 Apr. 19; 4:45. doi: 10.1186/1752-0509-4-45), and the Saccharomyces cerevisiae metabolic model iMM904 (Mo et al., BMC Syst Biol. 2009 Mar. 25; 3:37. doi: 10.1186/1752-0509-37). This model has been modified to include the improvements described according to the invention, including a heterologous CO₂ fixation pathway with (i) the addition of a PRK-type reaction, (ii) the addition of a RuBisCO-type reaction.

In particular exemplary embodiments, the reactions necessary to simulate the production of molecules through heterologous pathways have also been added to the model.

In a particular exemplary embodiment, a farnesene synthase reaction (EC 4.2.3.46 or EC 4.2.3.47) has been added for the heterologous production of farnesene.

In a second particular exemplary embodiment, acetoacetyl-CoA reductase (EC 1.1.1.36) and poly-β-hydroxybutyrate synthase (EC 2.3.1.B2 or 2.3.1.B5) reactions were added to the model to simulate a heterologous production pathway for β-hydroxybutyrate, the monomer of polyhydroxybutyrate. The simulations were carried out by applying to the model a set of constraints reproducible by the skilled person, aimed at simulating the in vivo culture conditions of a S. cerevisiae strain under the conditions described according to the invention (for example presence of unrestricted glucose in the medium, aerobic culture condition).

The simulations were carried out by applying to the model a set of constraints reproducible by the skilled person, aimed at simulating the in vivo culture conditions of a S. cerevisiae strain under the conditions described according to the invention (for example presence of unrestricted glucose in the medium, aerobic culture condition).

In particular exemplary embodiments, simulations are performed by virtually inactivating the reactions of the transaldolase enzymes TAL1 and NQM1, in order to simulate the decreases in activity of the non-oxidative branch of the pentose pathway, described according to the invention.

Simulations are carried out in parallel on an unmodified “wild-type strain” model in order to evaluate the impact of the improvements described according to the invention on the production yield of the biosynthetic pathways tested.

### ii) Results

The theoretical yields obtained and the percentages of improvement provided by the invention are described in Table 7 below.

TABLE 7
Maximum theoretical production yields evaluated by FBA on a wild-type strain and a modified
strain according to the invention, for the production of different molecules.

| | | | | | | | Percentage |
|---|---|---|---|---|---|---|---|
| | | | | Maximum theoretical production | | | improvement in |
| | Maximum theoretical production | | | yields with a modified strain | | | theoretical mass |
| | yields with a wild-type strain | | | according to the invention | | | yield g_{X}/g_{GLUC} |
| | Mol_{X}/ | CMol_{X}/ | g_{X}/ | Mol_{X}/ | CMol_{X}/ | g_{X}/ | provided by the |
| Target molecule | Mol_{GLUC} | CMol_{GLUC} | g_{GLUC} | Mol_{GLUC} | CMol_{GLUC} | g_{GLUC} | invention |
| Glutamate | 0.92 | 0.77 | 0.75 | 1 | 0.83 | 0.82 | +9.3% |
| β-Hydroxybutyric | 0.92 | 0.61 | 0.53 | 1 | 0.67 | 0.58 | +9.4% |
| acid | | | | | | | |
| Farnesene | 0.21 | 0.54 | 0.24 | 0.22 | 0.56 | 0.25 | +4.2% |

Mol_{X}/Mol_{GLUC}: moles of molecule X produced, in relation to the moles of glucose consumed
CMol_{X}/CMol_{GLUC}: moles of carbon of molecule X produced, in relation to the moles of carbon of glucose consumed
g_{X}/g_{GLUC}: g of molecule X produced, in relation to the g of glucose consumed

### Example 2: Improvement of Heterologous Farnesene Production in S. cerevisiae

A Saccharomyces cerevisiae yeast strain, CEN.PK 1605 (Mat a HISS leu2-3.112 trp1-289 ura3-52 MAL.28c) derived from the commercial strain CEN.PK 113-7D (GenBank: JRIV00000000) is engineered to produce NADPH without CO₂ loss and thus improve the production of farnesene alpha from glucose.

### a) Inactivation of the Non-Oxidative Branch of the Pentose Phosphate Pathway

The non-oxidative branch of the pentose phosphate pathway was inactivated by the deletion of the TAL1 gene and its paralogue NQM1.

### i) Inactivation of the TAL1 Gene: Chromosome XI (836350 to 837357, Complementary Strand)

To that end, the coding phase of the G418 resistance gene, derived from the KanMX cassette contained on plasmid pUG6 (P30114)—Euroscarf, was amplified with the oligonucleotides Sdtal1-Rdtal1 (Table 8).

TABLE 8
Oligonucleotides

| Name | sequence |
|---|---|
| Sdtal1 | ACGATAGTAAAATACTTCTCGAACTCGTCACATA |
| (SEQ ID NO: 1) | TACGTGTACATAATGGGTAAGGAAAAGACTCACG |
| | TTTC |
| Rdtal1 | ATCAAAAGAAACGTGCATAAGGACATGGCCTAAA |
| (SEQ ID NO: 2) | TTAATATTTCCGAGATACTTCCTTAGAAAAACTC |
| | ATCGAGCATCAAATGAAAC |
| Sdnqm1 | TTGCTAGCGTAAGTCATAAAAAATAGGAAATAAT |
| (SEQ ID NO: 3) | CACATATATACAAGAAATTAAATATGGGTAAAAA |
| | GCCTGAACTCACCG |
| Rdnqm1 | AGTGGTATATATATATTTATATATATAAGTAGGT |
| (SEQ ID NO: 4) | ACCTCTACTCTTAATGATTATTCCTTTGCCCTCG |
| | GACG |

The underlined portion of the oligonucleotides is perfectly homologous to the KanMX sequence and the rest of the sequence corresponds to the regions adjacent to the coding phase of the TAL1 gene on the Saccharomyces cerevisiae genome, so as to generate a PCR amplicon containing at its ends homologous recombination sequences of the TAL1 gene locus.

For the transformation reaction, strain CEN.PK 1605 was grown in a volume of 50 mL of complex rich medium YPD (yeast extract peptone dextrose) at 30° C., to an optical density at 600 nm of 0.8. The cells were centrifuged for 5 minutes at 2,500 rpm at room temperature. The supernatant was removed and the cells were resuspended in 25 mL of sterile water and centrifuged again for 5 minutes at 2,500 rpm at room temperature. After removing the supernatant, the cells were resuspended in 400 μL of 100 mM sterile lithium acetate.

At the same time, a transformation mix was prepared in a 2 mL tube as follows: 250 μL of 50% PEG, 10 μL of “carrier” DNA at 5 mg/mL, 36 μL of 1 M lithium acetate, 10 μL of purified PCR reaction (deletion cassette) and 350 μL of water.

The resuspended cells (50 μL) were added to the transformation mixture and incubated at 42° C. for 40 minutes in a water bath.

After incubation, the tube was centrifuged for 1 minute at 5,000 rpm at room temperature and the supernatant was discarded. The cells were resuspended in 2 mL of YPD, transferred to a 14 mL tube and incubated for 2 hours at 30° C. at 200 rpm. The cells were then centrifuged for 1 minute at 5,000 rpm at room temperature. The supernatant was removed and the cells were resuspended in 1 mL of sterile water and centrifuged again for 1 minute and resuspended in 100 μL of sterile water and spread over YPD+180 μg/mL G418.

The colonies obtained were genotyped for validation of the deletion of the TAL1 gene and referenced EQ-0520 (CEN.PK1605 Δtal1::kan).

### ii) Inactivation of the NQM1 Gene: Chromosome VII (580435 to 581436, Complementary Strand)

The coding phase of the hygromycin B resistance gene, derived from the hphMX cassette (loxP-pAgTEF1-hphMX-tAgTEF1-loxP) and contained on plasmid pUG75 (P30671)—Euroscarf, is amplified with the oligonucleotides Sdnqm1 and Rdnqm1 (Table 8). This generates a Δnqm1 PCR amplicon containing at its ends homologous recombination sequences of the transaldolase NQM1 gene locus.

For the transformation reaction, strain EQ-0520 (CEN.PK1605 Δtal1::kan) was grown in a 50 mL volume of complex rich medium YPD (yeast extract peptone dextrose) at 30° C. to an optical density at 600 nm of 0.8. The cells were centrifuged for 5 minutes at 2,500 rpm at room temperature. The supernatant was removed and the cells were resuspended in 25 mL of sterile water and centrifuged again for 5 minutes at 2,500 rpm at room temperature. After removing the supernatant, the cells were resuspended in 400 μL of 100 mM sterile lithium acetate. At the same time, a transformation mix was prepared in a 2 mL tube as follows: 250 μL of 50% PEG, 10 μL of “carrier” DNA at 5 mg/mL, 36 μL of 1 M lithium acetate, 10 μL of purified PCR reaction (deletion cassette) and 350 μL of water.

The resuspended cells (50 μL) were added to the transformation mixture and incubated at 42° C. for 40 minutes in a water bath. After incubation, the tube was centrifuged for 1 minute at 5,000 rpm at room temperature and the supernatant was discarded. The cells were resuspended in 2 mL of YPD, transferred to a 14 mL tube and incubated for 2 hours at 30° C. at 200 rpm. The cells were then centrifuged for 1 minute at 5,000 rpm at room temperature. The supernatant was removed and the cells were resuspended in 1 mL of sterile water and centrifuged again for 1 minute and resuspended in 100 μL of sterile water and spread on YPD+200 μg/mL hygromycin B+180 μg/mL G418.

The colonies obtained were genotyped for the validation of the deletion of the TAL1 gene and referenced EQ-0521 (CEN.PK1605 Δtal1::kan Δnqm1::hph).

### b) Introduction of PRK/RuBisCO/Farnesene Synthase Enzymes

In order to create an alternative pathway to glycolysis and allow strain EQ-0521 (CEN.PK1605 Δtal1::kan Δnqm1::hph) to increase the yield of certain metabolic products by fixing CO₂, the strain is modified to express:
a gene encoding a phosphoribulokinase PRK that grafts onto the pentose phosphate pathway by consuming ribulose-5P to give ribulose-1.5bisP and
a type I RuBisCO (with the structural genes RbcL and RbcS and the chaperones RbcX, GroES and GroEL). RuBisCO consumes ribulose-1.5bisP and one mole of CO₂ to form 3-phosphoglycerate

To produce alpha-farnesene, the alpha-farnesene synthase gene (AFS1; GenBank accession number AY182241) is missing from the yeast.

TABLE 9
Expression cassettes and plasmid composition

| | | Codon | | | | Auxotrophic | | | |
|---|---|---|---|---|---|---|---|---|---|
| Proteins | GenBank | optimization | Promoter | Terminator | ori | marker | Plasmids | | |
| RbcL | BAD78320.1 | Yes | TDH3p | ADH1 | 2μ | URA3 | pFPP45 | pL4 | |
| RbcS | BAD78319.1 | Yes | TEF1p | PGK1 | 2μ | URA3 | pFPP45 | pL4 | |
| RbcX | BAD80711.1 | Yes | TEF1p | PGK1 | ARS- | LEU2 | pFPP56 | | |
| | | | | | CEN6 | | | | |
| GroES | U00096 | No | PGI1p | CYC1 | ARS- | LEU2 | pFPP56 | | |
| | | | | | CEN6 | | | | |
| GroEL | AP009048 | No | TDH3 | ADH1 | ARS- | LEU2 | pFPP56 | | |
| | | | | | CEN6 | | | | |
| PRK | BAD78757.1 | Yes | Tet-OFF | CYC1 | ARS41 | TRP1 | pFPP20 | | |
| | | | | | 6-CEN4 | | | | |
| alpha- | AY182241 | Yes | PGI1p | CYC1 | 2μ | URA3 | | pL4 | pL5 |
| Farnesene | | | | | | | | | |
| synthase | | | | | | | | | |
| Empty | | | Tet-OFF | CYC1 | ARS41 | TRP1 | pCM185 | | |
| | | | | | 6-CEN4 | | | | |
| Empty | | | | | ARS- | LEU2 | pFL36 | | |
| | | | | | CEN6 | | | | |

The seven genes required for the engineering (Table 9) were cloned on three plasmid vectors capable of autonomous replication, with compatible origins of replication and each carrying a gene for supplementation of different auxotrophies, allowing the selection of strains containing the three plasmid constructs. Two of these plasmids are single-copy, with an Ars/CEN origin of replication and the third is multicopy with a 2μ origin.

Genes from Synechococcus elongatus, such as RbcL, RbcS, RbcX and PRK (previously described in WO 2015107496 A1) and alpha-farnesene synthase from Malus domestica (Tippmann et al. Biotechnol Bioeng. 2016 January; 113(1):72-81) have been optimized for the use of codons in Saccharomyces cerevisiae yeast.

According to the previously described protocol, strain EQ-0521 was grown in a volume of 50 mL of complex rich medium YPD at 30° C. and with the following transformation mix: 250 μL of 50% PEG, 10 μL of “carrier” DNA at 5 mg/mL, 36 μL of 1 M lithium acetate, 10 μL (3 μg of a combination of pFPP45+pFPP56+pFPP20 or pL4+pFPP56+pFPP20 or pL5+pFL36+pCM185) and 350 μL of water.

The resuspended cells (50 μL) were added to the transformation mixture and incubated at 42° C. for 40 minutes in a water bath. After incubation, the tube was centrifuged for 1 minute at 5,000 rpm at room temperature and the supernatant was discarded. The cells were resuspended in 2 mL YNB (yeast without nitrogen base supplemented with ammonium sulfate¹, glucose) supplemented with a commercial medium CSM (MP Biomedicals) suitable for selection markers, transferred into a 14 mL tube and incubated for 2 hours at 30° C. The final mix is spread on YNB+ammonium sulfate+CSM−LUW (leucine uracil, tryptophan in 20 g/L glucose and 2 μg/mL doxycycline.

The strains obtained are:
EQ-0523 (CEN.PK1605 Δtal1::kan Δnqm1::hph) (pFPP45+pFPP56+pFPP20)
EQ-0524 (CEN.PK1605 Δtal1::kan Δnqm1::hph) (pL4+pFPP56+pFPP20)
EQ-0525 (CEN.PK1605) (pL5+pFL36+pCM185)

Strains EQ-0523 (PRK/RuBisCO/Δtal1::kan Δnqm1::hph), EQ-0524 (PRK/RuBisCO/Δtal1::kan Δnqm1::hph+farnesene synthase) and EQ-0525 (farnesene synthase) to growth on liquid medium YNB with 20 g/L glucose and 10% CO₂

Batch-mode cultures in Erlenmeyer flasks are carried out with the appropriate culture medium and a 10% exogenous CO₂ supply, in a shaking incubator (120 rpm, 30° C.), with an inoculation at 0.05 OD600 nm measured using an EON spectrophotometer (BioTek Instruments). The strain of interest is grown on YNB+CSM-LUW medium with 20 g/L glucose and a 10% exogenous CO₂ supply.

After observation of a significant growth start, the strains are adapted to a minimum mineral medium free of amino acids and nitrogenous base included in the CSM-LUW, i.e. only YNB with 20 g/L glucose and a 10% exogenous CO₂ supply c) Production of farnesene in Erlenmeyer flasks

Saccharomyces cerevisiae strain EQ-0524, whose non-oxidative branch of the pentose phosphate pathway is inhibited by inhibition of the TAL1 and NQM1 genes, is grown in order to produce farnesene by overproducing NADPH without CO₂ loss, using exogenous PRK and RuBisCO. This strain of interest is compared with a reference strain EQ-0525 producing farnesene following the addition of a heterologous farnesene synthase, without deletion of TAL1 and NQM1 or addition of exogenous PRK and RuBisCO. Batch-mode cultures in Erlenmeyer flasks are carried out under the conditions described above.

The farnesene concentration is quantified from the supernatant of fermentation must. Briefly, the cell suspensions are centrifuged at 5000 rpm for 5 minutes. The dodecane phase is diluted 10 times in hexane and injected into GC-MS, for analysis according to the protocol described in Tippman et al. (Biotechnol Bioeng. 2016; 1131:72-81).

A 3% increase in production yield, in grams of farnesene per gram of glucose consumed, was observed in strain EQ-0253, compared with strain EQ-0253

### Example 3: Improvement of Glutamate Production in E. coli

It has already been described that deletion of the alpha-ketoglutarate dehydrogenase gene increases glutamate production (Usuda et al., J Biotechnol. 2010 May 3; 147(1):17-30. doi: 10.1016/j.jbiotec.2010.02.018). The experiments described below were therefore carried out in an Escherichia coli K12 strain MG1655, whose sucA gene has been deleted. This strain is derived from a gene deletion bank (Baba et al., Mol Syst Biol. 2006; 2:2006.0008) in Escherichia coli and supplied by the Coli Genetic Stock Center under the name JW0715-2 and with reference 8786. (JW0715-2: MG1655 ΔsucA::Kan).

### a) Removal of the Selection Cassette by Specific Recombination of FTR Regions by Flp Recombination

In order to be able to reuse the same deletion strategy as that used to construct strain JW0715-2 above, the selection cassette had to be removed, using a recombinase.

Plasmid p707-Flpe (provided in the Quick & Easy E. coli Gene Deletion Red®/ET® Recombination Kit by Gene Bridges) is electroporated according to the kit protocol. The cells are selected on LB agar supplemented with 0.2% glucose, 0.0003% tetracycline and added with 0.3% L-arabinose. A counter-selection of the clones obtained is carried out by verifying that they are no longer able to grow on the same medium supplemented with 0.0015% kanamycin.

The strain obtained is called EQ.EC002: MG1655 ΔsucA

b) Deletion of the edd-eda Operon, Encoding the Entner-Doudoroff Metabolic Pathway

The deletion of the edd-eda operon is performed by homologous recombination and use of the Quick & Easy E. coli Gene Deletion Red®/ET® Recombination Kit (Gene Bridges) according to the supplier's protocol.
1. Oligonucleotides designed to amplify an FRT-PKG-gb2-neo-FRT resistance gene expression cassette and having a 5′ sequence homologous, over 50 nucleotides, to adjacent regions of the deletion locus (positions 1932065-1932115 and 1934604-1934654) on the chromosome, thus generating recombination arms of the cassette on the bacterial genome on either side of the entire operon;
2. The Escherichia coli K-12 strain EQ.EC002 is transformed by electroporation with plasmid pRedET according to the kit protocol. The colonies obtained are selected on rich complex medium LB agar with 0.2% glucose, 0.0003% tetracycline;
3. Transformation of the amplicon obtained in the first step in the presence of RedET recombinase, induced by 0.3% arabinose in liquid LB for 1 h. To that end, a second electroporation of the cells expressing RedET by the deletion cassette is performed and the colonies are selected on LB agar supplemented with 0.2% glucose, 0.0003% tetracycline and added with 0.3% L-arabinose and 0.0015% kanamycin.
4. Plasmid p707-Flpe (provided in the Quick & Easy E. coli Gene Deletion Red®/ET® Recombination Kit by Gene Bridges) is transformed by electroporation according to the kit protocol. The cells are selected on LB agar supplemented with 0.2% glucose, 0.0003% tetracycline and added with 0.3% L-arabinose. A counter-selection of the clones obtained is carried out by verifying that they are no longer able to grow on the same medium supplemented with 0.0015% kanamycin.
5. The strain obtained is called EQ.EC003: MG1655 ΔsucA Δedd-eda
   c) Deletion of the talA Gene

The deletion of the talA gene is performed by homologous recombination and the use of the Quick & Easy E. coli Gene Deletion Red®/ET® Recombination Kit (Gene Bridges) according to the supplier's protocol.
1. Oligonucleotides designed to amplify an FRT-PKG-gb2-neo-FRT resistance gene expression cassette and having a 5′ sequence homologous over 50 nucleotides to the adjacent regions of the deletion locus, i.e. the coding phase of the gene (talA) (Gene ID: 947006), thus generating recombination arms of the cassette on the bacterial genome.
2. The Escherichia coli K-12 strain EQ.EC003 is transformed by electroporation with plasmid pRedET, according to the kit protocol. The colonies obtained are selected on rich complex medium LB agar with 0.2% glucose, 0.0003% tetracycline.
3. Transformation of the amplicon obtained in the first step, in the presence of the RedET recombinase which will be induced by 0.3% arabinose in liquid LB for 1 h. To that end, a second electroporation of the cells expressing RedET by the deletion cassette is performed and the colonies are selected on LB agar supplemented with 0.2% glycerol and 0.3% pyruvate, 0.0003% tetracycline and added with 0.3% L-arabinose and 0.0015% kanamycin.

Deletions are verified by genotyping and sequencing and the name of the strains obtained is
EQ.EC002: MG1655 ΔsucA
EQ.EC003: MG1655 ΔsucA Δedd-eda
EQ.EC020: MG1655 ΔsucA Δedd-edda ΔtalA::kan

### d) Insertion of PRK/RuBisCO Engineering for CO₂ Fixation

For the recombinant expression of the different components of a type I RuBisCO in E. coli, the genes described in the

Table below are cloned as a synthetic operon containing the genes described in Table below.

To control the expression level of these genes, ribosome binding sequences (RBS) presented in the

Table, with varying translation efficiencies, as described in Zelcbuch et al. (Zelcbuch et al., Nucleic Acids Res. 2013 May; 41(9):e98; Levin-Karp et al., ACS Synth Biol. 2013 Jun. 21; 2(6):327-36. doi: 10.1021/sb400002n) are inserted between the coding phase of each gene. The succession of each coding phase interspersed by an RBS sequence is constructed by successive insertion into a pZA11 vector (Expressys) that contains a PLtetO-1 promoter, a p15A origin of replication, and an ampicillin resistance gene.

TABLE 10
Gene references

| Genes | GenBank | Organism |
|---|---|---|
| rbcL | BAD78320.1 | Synechococcus elongatus |
| rbcS | BAD78319.1 | Synechococcus elongatus |
| rbcX | BAD80711.1 | Synechococcus elongatus |
| Prk | BAD78757.1 | Synechococcus elongatus |

TABLE 11
Composition of expression cassettes on plasmids

| | Structure of the synthetic operon in vector pZA11 | | | | | | | | |
|---|---|---|---|---|---|---|---|---|---|
| Plasmid | geneA | RBS1 | geneB | RBS2 | geneC | RBS3 | geneD | RBS4 | geneE |

| | | | | | | | |
|---|---|---|---|---|---|---|---|
| pZA11 | | | | | | | |
| pEQEC005 | rbcS | D | rbcL | B | RbcX | F | |
| pEQEC006 | rbcS | D | rbcL | B | RbcX | F | Prk |
| pEQEC008 | Prk | | | | | | |

TABLE 12
Ribosome binding site (RBS)
intercistronic sequences

| Name | RBS sequences |
|---|---|
| A (SEQ ID NO: 5) | AGGAGGTTTGGA |
| B (SEQ ID NO: 6) | AACAAAATGAGGAGGTACTGAG |
| C (SEQ ID NO: 7) | AAGTTAAGAGGCAAGA |
| D (SEQ ID NO: 8) | TTCGCAGGGGGAAG |
| E (SEQ ID NO: 9) | TAAGCAGGACCGGCGGCG |
| F (SEQ ID NO: 10) | CACCATACACTG |

Several strains are produced by electroporating the different vectors presented according to the plan above:

EQ.EC 020→(EQ.EC 003+pZA11): MG1655 ΔsucA Δedd-eda

EQ.EC 021→(EQ.EC 004+pEQEC005): MG1655 ΔsucA Δedd-eda-talA::kan (RuBisCO)

EQ.EC 022→(EQ.EC 004+pEQEC006): MG1655 ΔsucA Δedd-eda talA::kan (RuBisCO+PRK)

EQ.EC 024→(EQ.EC 003+pEQEC008): MG1655 ΔsucA Δedd-eda ΔtalA::kan (PRK)

Clones are selected on LB medium supplemented with 100 mg/L ampicillin. After obtaining a sufficient quantity of biomass, cultures with a volume greater than or equal to 50 mL in Erlenmeyer flasks of at least 250 mL are inoculated in order to adapt the strain to the use of the PRK/RuBisCO engineering. This adaptation is carried out on LB culture medium with 2 g/L glucose, and an exogenous CO₂ supply of 1 atmosphere at 37° C. as described above.

### e) Glutamate Production

For glutamate production, cells from 500 mL of LB culture are inoculated into 20 mL of MS medium (40 g/L glucose, 1 g/L MgSO⁴⁻.7H₂O, 20 g/L (NH₄)₂SO₄, 1 g/L KH₂PO₄, 10 mg/L FeSO₄.7H₂O, 10 mg/L MnSO₄.7H₂O, 2 g/L yeast extract, 30 g/L CaCO₃, 100 mg/L ampicillin at a pressure of 0.1 atmosphere CO₂.

Residual glutamate and glucose are measured with a bioanalyzer (Sakura Seiki). The carbon yield Y_{p/s} is calculated in grams of glutamate produced per gram of glucose consumed. This yield increases by 8% in EQ.EC 022 strains (RuBisCO+PRK), compared with the control strains EQ.EC 020 (empty), EQ.EC 021 (RuBisCO only). The control strain EQ.EC 024 (PRK alone) is not viable.

### Example 4: Improvement of PHB Production in C. Necator

### a) Inhibition of the Non-Oxidative Branch of the Pentose Phosphate Pathway

Increasing the reducing power can also significantly improve the efficiency of existing metabolic pathways. This is the case for the bacterial strain Ralstonia eutropha ATCC 17699 (Cupriavidus necator) which naturally produces polyhydroxybutyrate (PHB). This bacterium is capable of developing under both autotrophic and heterotrophic conditions.

The deletion, according to the invention, of the tal gene (Transaldolase MF_00492) makes it possible to concentrate the metabolic flux on the oxidative pentose phosphate pathway, by increasing the pool of NADPH-reduced nucleotides, thus increasing the PHB production yield but also allowing the use of the glycolysis pathway.

This Cupriavidus necator strain (R. eutropha H16) has a megaplasmid pHG1 and two chromosomes. The deletion of the tal gene is achieved by generating a vector containing a SacA suicide gene for Gram-negative bacteria, as described in Quandt et al. and Lindenkamp et al. (Quandt et al., Gene. 1993 May 15; 127(1):15-21; Lindenkamp et al., Appl Environ Microbiol. 2010 August; 76(16):5373-82; Lindenkamp et al., Appl Environ Microbiol. 2012 August; 78(15):5375-83).

Two PCR amplicons corresponding to adjacent regions of the tal gene are cloned by restriction according to the procedure described in Lindenkamp et al. (Appl About Microbiol. 2012 August; 78(15):5375-83) in plasmid pjQ200mp18Tc. The modified plasmid pjQ200mp18Tc::Δtal is then transformed into an E. coli strain S17-1, by the calcium chloride transformation method. And the transfer of genetic material is done by conjugation by depositing on agar a spot of Ralstonia Eutropha culture on a dish containing a cell monolayer of S17-1 bacteria and selection is made on nutrient broth (NT) medium at 30° C., in the presence of 10% sucrose for purposes of selection (Hogrefe et al., J Bacteriol. 1984 April; 158(1):43-8.) and validated on a mineral medium containing 25 μg/mL tetracycline.

The deletions are validated by genotyping and sequencing. The resulting strain EQCN_002 therefore has a deletion of the tal gene. EQCN_010: H16 Δtal

### b) Inactivation of the Entner-Doudoroff Metabolic Pathway

Two PCR amplicons corresponding to adjacent regions of the edd and eda genes (upstream of edd and downstream of eda) are cloned by restriction according to the procedure described in Srinivasan et al. (Appl Environ Microbiol. 2002 December; 68(12):5925-32) in plasmid pJQ200mp18Cm.

The modified plasmid pJQ200mp18Cm::Δedd-eda is then transformed into an E. coli strain S17-1, by the calcium chloride transformation method. And the transfer of genetic material is done by conjugation, by depositing on agar a spot of Ralstonia eutropha EQCN_010 culture on a dish containing a cell monolayer of S17-1 bacteria and the selection is made on nutrient broth (NT) medium at 30° C., in the presence of 10% sucrose for purposes of selection (Hogrefe et al., J Bacteriol. 1984 April; 158(1):43-8.) and validated on a mineral medium containing 50 μg/mL chloramphenicol.

The deletions are validated by genotyping and sequencing. The resulting strain EQCN_003 therefore has a deletion of the tal gene. EQCN_011: H16 Δtal Δedd-eda

### c) Production of PHB in a Bioreactor

The inoculum from a frozen stock is spread on solid medium at a rate of 50 to 100 μL from a cryotube incubated at 30° C. for 48 to 96 h in the presence of glucose. The expression of genes encoding RuBisCO and PRK are maintained in C. necator under heterotrophic aerobic conditions (Rie Shimizu et al., Sci Rep. 2015; 5: 11617. Published online 2015 Jul. 1.). Batch-mode cultures in Erlenmeyer flasks (10 mL in 50 mL, then 50 mL in 250 mL) are carried with the appropriate culture medium, in 20 g/L glucose and a 10% exogenous CO₂ supply in a shaking incubator (100-200 rpm, 30° C.), with a minimum inoculation of 0.01 OD₆₂₀ₙₘ.

The strain of interest EQCN_011 improving PHB production yield is compared with a reference strain H16 naturally accumulating PHB under heterotrophic conditions in the presence of a nutritional limitation.

The productivity of the strains is compared in bioreactors. Cultures carried out in bioreactors are seeded from solid and/or liquid amplification chains in Erlenmeyer flasks, under the conditions described above. The bioreactors, of type My-control (Applikon Biotechnology, Delft, Netherlands) 750 mL or Biostat B (Sartorius Stedim, Göttingen, Germany) 2.5 L, are seeded at a minimum concentration equivalent to 0.01 OD₆₂₀ₙₘ.

The accumulation of PHB is decoupled from growth. The culture is regulated at 30° C., aeration is maintained between 0.1 VVM (gas volume/liquid volume/min) and 1 VVM, in order to maintain a minimum dissolved oxygen concentration above 20% (30° C., 1 bar). Shaking is adapted according to the scale of the bioreactor used. The inlet gas flow consists of air optionally supplemented with CO₂. CO₂ supplementation is between 1% and 10%. The pH is adjusted to 7 by adding a 14% or 7% ammonia solution. The fed-batch culture method allows a supply of non-limiting carbon substrate combined with a limitation of phosphorus or nitrogen, while maintaining a constant carbon/phosphorus or carbon/nitrogen ratio.

PHB extraction and quantification are performed according to the method of Brandl et al. (Appl Environ Microbiol. 1988 August; 54(8):1977-82.)

The protocol consists in adding 1 mL of chloroform to 10 mg of lyophilized cells followed by 850 μL of methanol and 150 μL of sulfuric acid. The mixture is heated for 2.5 hours at 100° C., cooled and 500 μL of water is added. The two phases are separated by centrifugation and the organic phase is dried by adding sodium sulfate.

The samples are filtered and analyzed as described by Müller et al. (Appl Environ Microbiol. 2013 July; 79(14):4433-9). Comparison of the cultures of wild-type C. necator H16 and of strain EQCN_011: H16 Δtal Δedd-eda, respectively, shows a 2% increase in PHB production yield (in grams of PHB per gram of glucose consumed) in favor of the modified strain according to the invention.

1. A genetically modified microorganism for the production of an exogenous molecule of interest and/or to overproduce an endogenous molecule of interest, other than a RuBisCO enzyme and/or phosphoribulokinase (PRK), said microorganism expressing a functional RuBisCO enzyme and a functional phosphoribulokinase (PRK), and in which the non-oxidative branch of the pentose phosphate pathway is at least partially inhibited, wherein said microorganism is genetically modified so as to produce an exogenous molecule of interest and/or to overproduce an endogenous molecule of interest, other than a RuBisCO enzyme and/or phosphoribulokinase (PRK).

2. The genetically modified microorganism according to claim 1, wherein said microorganism is genetically modified to express a recombinant RuBisCO enzyme and/or PRK.

3. The genetically modified microorganism according to claim 1, wherein said microorganism being genetically modified to inhibit the non-oxidative branch of the pentose phosphate pathway downstream of ribulose-5-phosphate production.

4. The genetically modified microorganism according to claim 1, wherein the expression of the gene encoding a transaldolase (E.C.2.2.1.2) and/or a transketolase (E.C.2.2.1.1) is at least partially inhibited.

5. The genetically modified microorganism according to one claim 1, wherein the exogenous molecule of interest and/or the endogenous molecule of interest is selected from amino acids, peptides, proteins, vitamins, sterols, flavonoids, terpenes, terpenoids, fatty acids, polyols and organic acids.

6. The genetically modified microorganism according to claim 1, said microorganism being a eukaryotic cell or a prokaryotic cell.

7. The genetically modified microorganism according to claim 1, wherein said microorganism is a yeast of the genus Saccharomyces cerevisiae genetically modified to express a functional type I or II RuBisCO and a functional phosphoribulokinase (PRK), and in which the expression of the TAL1 and/or NQM1 genes is at least partially inhibited.

8. (canceled)

9. A biotechnological process for producing at least one molecule of interest other than a RuBisCO enzyme and/or a phosphoribulokinase (PRK), wherein it comprises a step of culturing a genetically modified microorganism as defined in claim 1, under conditions allowing the synthesis or bioconversion, by said microorganism, of said molecule of interest, and optionally a step of recovering and/or purifying said molecule of interest.

10. The biotechnological process according to claim 9, wherein the microorganism is genetically modified to express at least one enzyme involved in the bioconversion or synthesis of said molecule of interest.

11. The biotechnological process according to claim 9, wherein the microorganism is genetically modified to at least partially inhibit an enzyme involved in the degradation of said molecule of interest.

12- A method for producing a molecule of interest other than a RuBisCO enzyme and/or a phosphoribulokinase (PRK), comprising (i) inserting at least one sequence encoding an enzyme involved in the synthesis or bioconversion of said molecule of interest into a recombinant microorganism as defined in claim 1, (ii) culturing said microorganism under conditions allowing the expression of said enzyme and optionally (iii) recovering and/or purifying said molecule of interest.

13. A method for producing a molecule of interest other than a RuBisCO enzyme and/or a phosphoribulokinase (PRK), comprising (i) inhibiting the expression of at least one gene encoding an enzyme involved in the degradation of said molecule of interest in a recombinant microorganism as defined claim 1, (ii) culturing said microorganism under conditions allowing the expression of said enzyme and optionally (iii) recovering and/or purifying said molecule of interest.

14. The genetically modified microorganism of claim 1, wherein it is an eukaryotic cell selected from yeasts, fungi and microalgae.

15. The genetically modified microorganism of claim 1, wherein it is a bacterium.
